(19) United States
(12) Patent Application Publication
(10) Pub. No.: US 2022/0254491 A1
(43) Pub. Date: Aug. 11, 2022

(54) HEALTH SAVINGS MANAGEMENT SYSTEM

(71) Applicant: Ming-Chia Hsieh, Taichung City (TW)

(72) Inventor: Ming-Chia Hsieh, Taichung City (TW)

(21) Appl. No.: 17/170,749

(22) Filed: Feb. 8, 2021

(51) Int. Cl. G16H 50/20; G16H 10/60

(52) U.S. Cl. CPC G16H 50/20; G16H 10/60

(57) ABSTRACT

A health savings management system installed and downloaded to a mobile device. The system includes an identity recording module, for recording gender, age and life deposit value. The identity recording module has a built-in predetermined age. A calculation module for performing a calculation based on the predetermined age and the inputted gender, age and life deposit value to obtain a total health credit limit and display the total health credit limit. A deduction module has a built-in and predetermined comparison standard value, and records physical condition, sickness, dietary habit, and rest, and comparing with the comparison standard value and then generating a deduction value. The calculation module adds up the predetermined age and the total health credit limit to change the numerical value of a total health credit limit according to the deduction value generated by the deduction module.

### BACKGROUND OF THE INVENTION

### 1. Fields of the Invention

The present invention relates to a health savings management system, which relates to technologies applied in the medical field, and mainly focuses on digitalization to health, and the concept of savings is used to effectively manage the health of the body, providing users with practical digital presentation to understand their physical condition.

### 2. Descriptions of Related Art

Health has always been the most important aspect of life to everyone, and many people regularly go to a hospital or medical center for physical checkups, and most physical checkup results and pathological reports are usually explained by doctors. However, the relevant explanations given by the doctors are sometimes too difficult to understand, especially those for the top ten diseases, because most people do not really understand various types of cancer or chronic diseases, and cancer cannot be predicted or prevented. On the other hand, chronic diseases can be prevented and treated, but the doctors' explanation is too difficult to understand, so that more and more people are making their own judgment whether they need to see a doctor. In the end, they gradually ignore the physical checkup and examination. However, some chronic diseases do not cause any discomfort, and the long-term neglect will only accelerate the damage to physical health, and more importantly, will delay the golden time of treatment, and finally will cause an irreparable consequence. Therefore, manufacturers of the related industry must think about how to improve the aforementioned problems and let patients feel the need of physical checkup and healthcare for their diseases.

The present invention intends to provide a health savings management system to eliminate the shortcomings mentioned above.

### SUMMARY OF THE INVENTION

The present invention relates to a health savings management system which is installed and downloaded to a mobile device for a user's review. The mobile device includes an input part and an output part. The health savings management system comprises an identity recording module for recording gender, age and life deposit value input through the input part by the user. The identity recording module has a built-in predetermined age. A calculation module for performing a calculation based on the predetermined age and the input gender, age and life deposit value to obtain a total health credit limit, and display the total health credit limit on the output part. A deduction module includes a built-in and predetermined comparison standard value, a deduction module for recording physical condition, sickness, dietary habit, and rest input by the user through the input part and comparing the inputted physical condition, sickness, dietary habit, and rest with the comparison standard value so as to generate a deduction value. The calculation module adds up the predetermined age and the total health credit limit to change the numerical value of the total health credit limit according to the deduction value generated by the deduction module. The user's physical health is expressed in terms of actual money by the health savings management system to facilitate the user to better understand her/his physical condition and pay attention to and strengthen healthcare.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 shows the components involved in the health savings management system of the present invention;

FIG. 2 shows that the health savings management system of the present invention is used with a mobile device;

FIG. 3 shows the display of the identity recording module of the health savings management system of the present invention;

FIG. 4 shows the display of the health deduction unit;

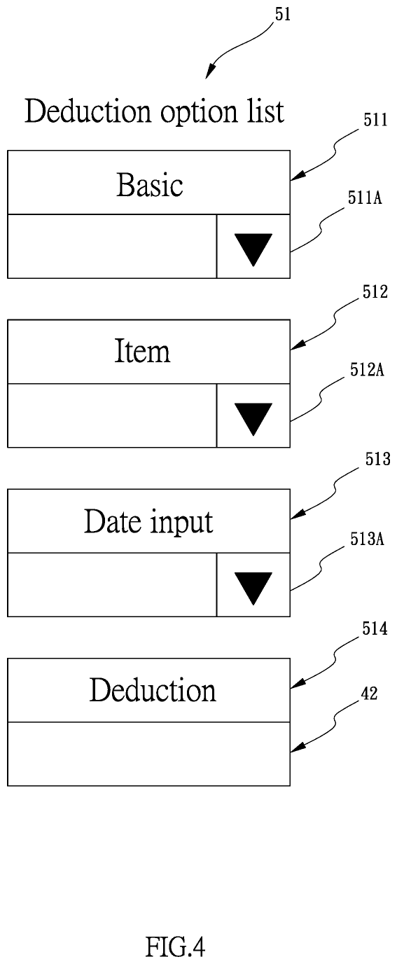

FIG. 5 shows the display of the basic field;

FIG. 6 shows the selecting specific scientific name of a medical test displayed of the option field;

FIG. 7 shows the display of the data input field;

FIG. 8 shows the display of the diseases name filed of the medical history record deduction unit;

FIG. 9 shows the display of the breakdown field of the disease name field;

FIG. 10 shows the display of the life column and physical exercise column of the habit item field;

FIG. 11 shows the display of the physical menu of the range column;

FIG. 12 shows the display the input record list of the savings deposit field, and

FIG. 13 shows the warning window of the input record list.

### DETAILED DESCRIPTION OF THE PREFERRED EMBODIMENT

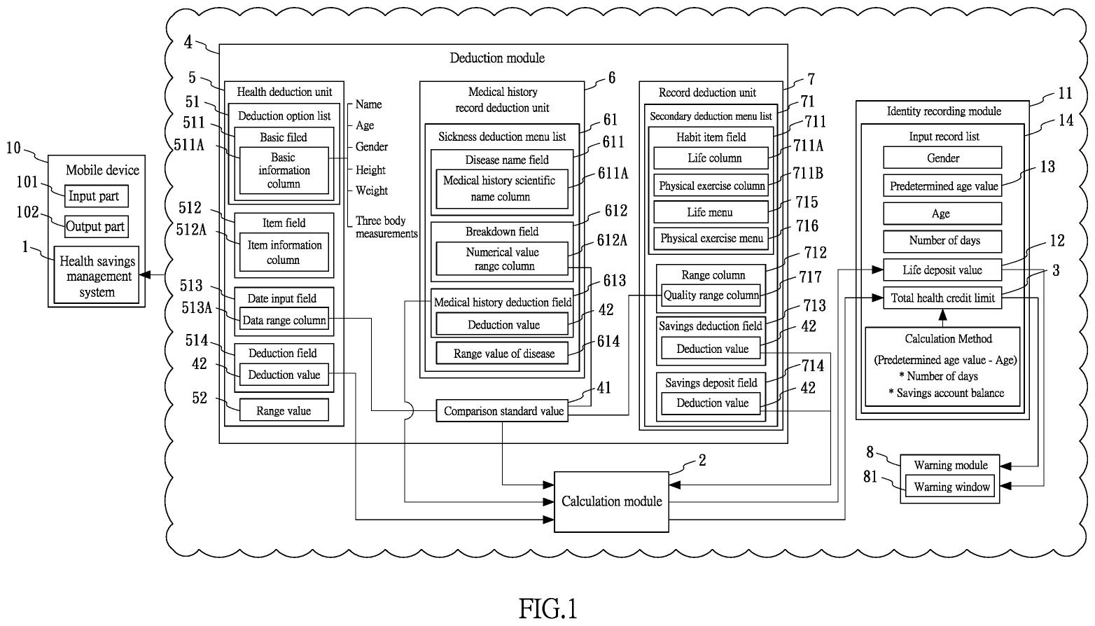

Referring to FIGS. 1 to 3, the health savings management system 1 of the present invention is installed and downloaded to a mobile device 10 and provided for a user's review. The mobile device 10 includes an input part 101 and an output part 102. The health savings management system 1 comprises an identity recording module 11 for recording gender, age and life deposit value 12 input through the input part 101 by the user. The identity recording module 11 has a built-in predetermined age 13. The identity recording module 11 comprises an input record list 14 which comprises a plurality of input fields 141. Each input field 141 is filled with gender, and age input by the user, a number of days and a life deposit value to be calculated. One of the input fields 141 is age which refers to the user's age value. Another input field 141 is the number of days which refers to the number of days required to be managed and recorded. Yet another input field 141 is the life deposit value 12 which refers to the monetary value required to be saved daily after health is converted into money. A calculation module 2 for performing a calculation based on the predetermined age 13 and the input gender, age and life deposit value 12 to obtain a total health credit limit 3 and to display the total health credit limit on the output part 102. The total health credit limit 3 is calculated by (Predetermined age 13 −Age)*Number of days*Life deposit value). The life deposit value 12 and the total health credit limit 3 are represented by the virtualization of health by money. The monetary value of the life deposit value 12 represents the user daily health amount. The monetary value of the total health credit limit 3 represents the user's total health value 3 from the numerical value of the predetermined age 13 to the numerical value of the age, which are provided for reference by the user in order to pay attention to health care. A deduction module 4 has a built-in and predetermined comparison standard value 41. The deduction module 4 records physical condition, sickness, dietary habit and rest input by the user through the input part 101. The inputted physical condition, sickness, dietary habit and rest are compared with the comparison standard value 41, and generates a deduction value 42. The calculation module 2 adds up the predetermined age 13 and the total health credit limit 3 to change the numerical value of the total health credit limit 3 according to the deduction value 42 generated by the deduction module 4. The user's physical health is expressed in terms of actual money by the health savings management system 1 to facilitate the user to better understand her/his own physical condition and pay attention to and strengthen healthcare.

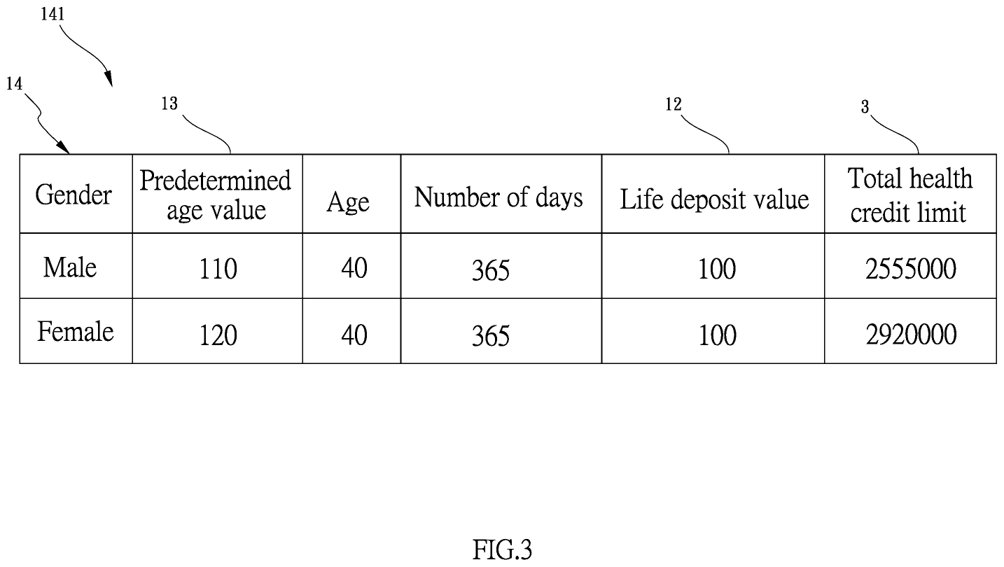

The user can input information into the identity recording module 11, as shown in FIG. 3 as an example, the user chooses male/female in the column 141. The total health credit limit 13 is automatically generated according to 110 year-old male/120 year-old female (110 year-old male/120 year-old female is an estimate value). One of the input fields 141 is the user's age value which is 40 years old male/female. Another input field 141 is the number of days which refers to the number of days required to be managed and recorded. Yet another input field 141 is 100 dollars which is the life deposit value 12 which refers to the monetary value required to be saved daily for the male/female after health is converted into money. The total health credit limit 3 is calculated by (Predetermined age−Age)*Number of days*Life deposit value), the life deposit value 12 is 2555000 dollars for male, and 2920000 dollars for female. A basic amount of money is obtained after filling all of the information mentioned above. For the male, the value of the total health credit limit 3 is 2555000 dollars.

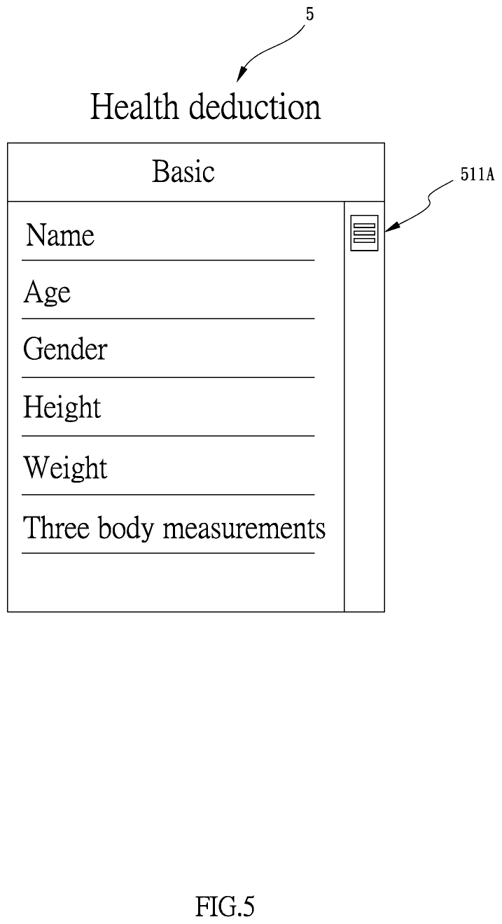

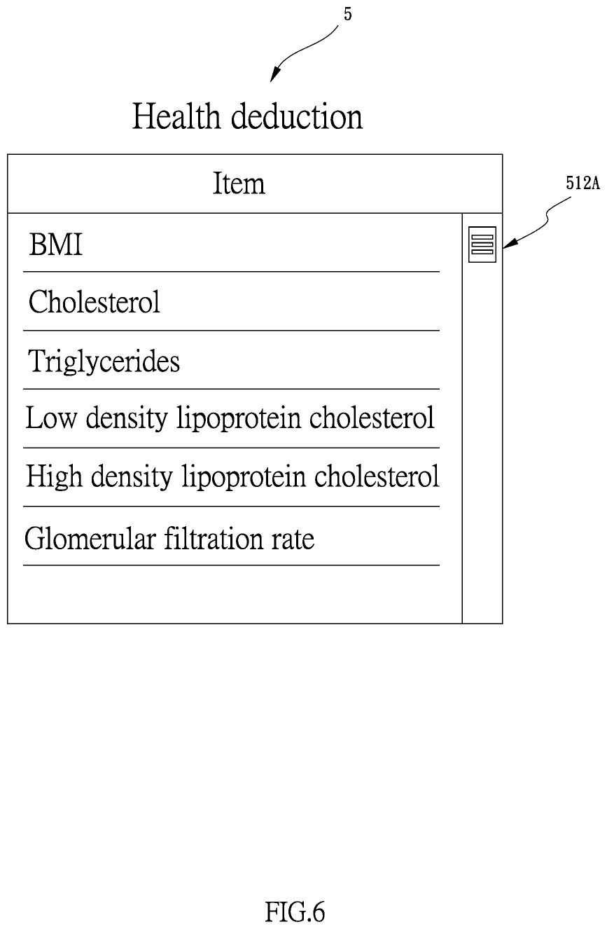

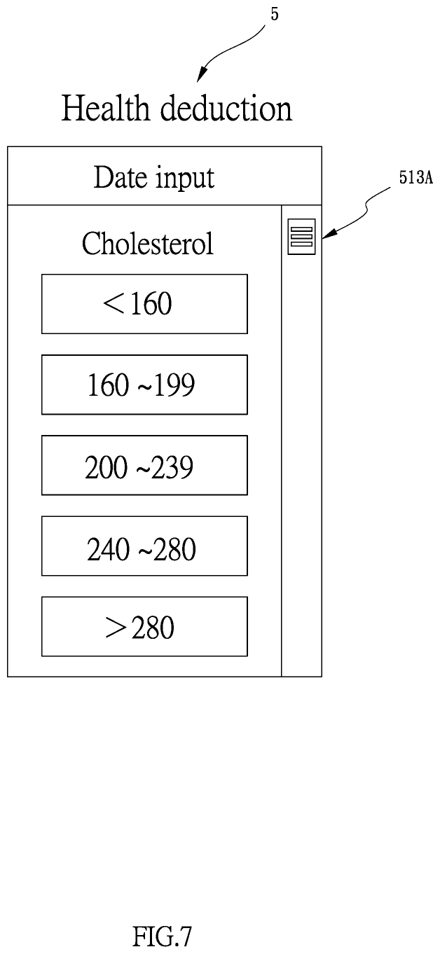

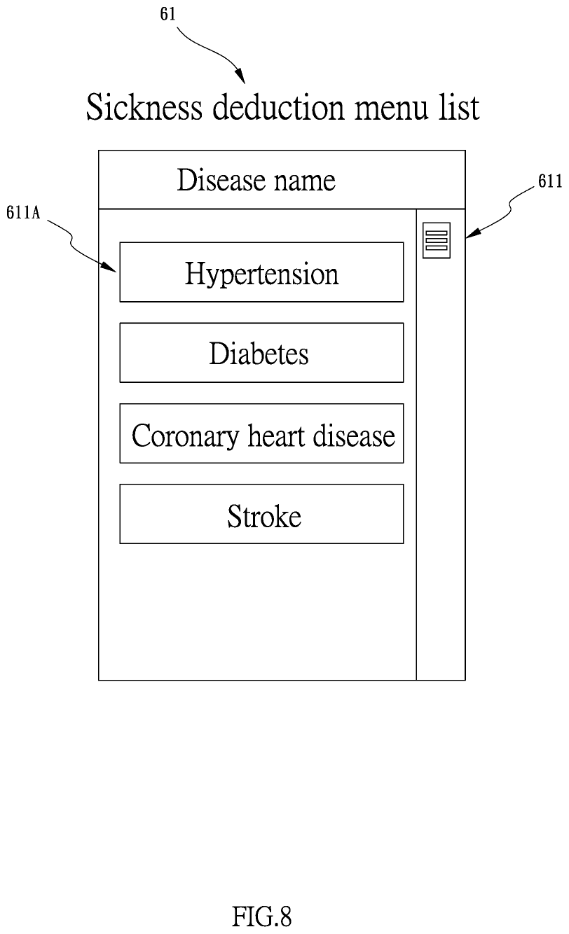

The user may further define the health situations by calculating the daily health amount. The deduction module 4 comprises a health deduction unit 5. The user inputs the data according to his/her health situation. Take the health deduction unit 5 as an example, the health deduction unit 5 has a deduction option list 51 which comprises a basic field 511, an item field 512, a data input field 513 and a deduction field 514. The basic field 511 is provided for inputting a user's basic physical information, the item field 512 is provided for inputting and selecting names of a medical test. The data input field 513 is provided for inputting the data of the name of the medical test input in the item field 512. The standard numerical value of the comparison standard value 41 is a constant built in the health savings management system 1. The data input into the data input field 513 is compared with the comparison standard value 41. The calculation module 2 is provided for calculating the deduction value 42 generated in the deduction field 514. The deduction value 42 generated in the deduction field 514 is added to the predetermined age 13 to obtain the user's total health credit limit 3, so as to check the user's physical condition. When filling information in the basic field 511 of the deduction option list 51 as shown in FIG. 5, the basic field 511 comprises a basic information column 511A for inputting the user's name, age, gender, height, weight and three body measurements by the input part 101. After finishing the basic filed 511, the user continues to fill the item filed 512 which comprises an item information column 512A as shown in FIG. 6. The item information column 512A is provided for inputting a scientific name of a specific sickness of the medical health examination by the input part 101. The item information column 512A has a plurality of options such as BMI, cholesterol, triglycerides, low density lipoprotein cholesterol, high density lipoprotein cholesterol and glomerular filtration rate. After finishing the item information column 512A, the data input field 513 pops up and comprises a data range column 513A as shown in FIG. 7. For example, the user chooses cholesterol, there are five ranges displayed in the data range column 513A, which are <160, 160-199, 200-239, 240-280, and >280. The values shown are the value of cholesterol. When the user chooses the value of cholesterol from the five ranges, the data input field 513 generates the data range column 513A from which the user chooses one. The health deduction unit 5 presets a plurality of range values 52. Each range value 52 presets the corresponding deduction value 42. After the data range column 513A selected from the data input field 513 is compared with the comparison standard value 41, the calculation module 2 determines if the numerical value determining the aforementioned comparison which falls within any range of each range value 52. The calculation module 2 adds the predetermined age 13 to the deduction value 42 to form the maximum value of the user's predetermined age 13. In addition, the above five range intervals are respectively set to correspond to a value to be deduction, that is, the deduction value 42. FIG. 8 illustrates the example when the cholesterol value measured by the user is 223, and the data range column 513A judges that this value falls in the range of 200-239. The user then select the above range. After selection, the calculation module 2 judges the above range and compares it with the comparison standard value 41, and then the deduction field 514 displays the value of minus 2 at the same time. The input record list 141 with the predetermined age of 13 in the record table 14 will be added to the value of minus 2, so that the maximum limit of the predetermined age 13 on the user side is changed to 108. The total amount of the total health credit limit 3 will also be changed to 24820000 dollars. By this way, the user is reminded to pay attention to physical health, and virtual money will be used to give the user a more sense of health.

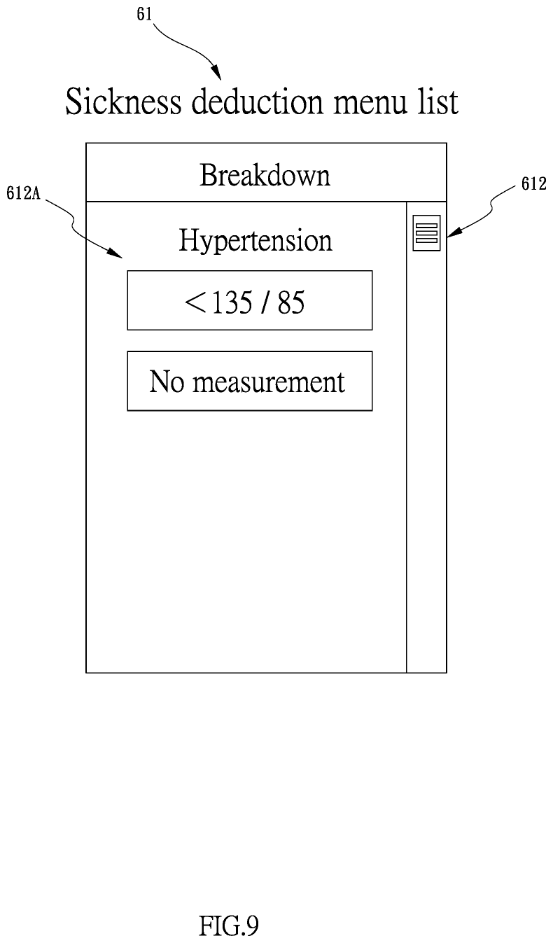

The deduction module 4 comprises a medical history record deduction unit 6 which allows the user to input the medial history. The medical history record deduction unit 6 and the identity recording module 11 let the user to pay attention to his/her health condition. As shown in FIG. 9, the medical history record deduction unit 6 comprises a sickness deduction menu list 61 which comprises a disease name field 611, a breakdown field 612 and a medical history deduction field 613. The user can choose one of the disease names provided in the disease name field 611 which has a medical history scientific name column 611A. The medical history scientific name column 611A is provided for inputting the user's sickness by the input part 101. The medical history scientific name column 611A comprises the disease names including hypertension, diabetes, coronary heart disease or stroke. Take the user having high blood pressure as an example. After selecting high blood pressure in the medical history scientific name column 611A, the medical history deduction field 613 calculates. Before the sum is obtained, after the user selects the high blood pressure, a breakdown field 612 pops in the value range field 612A which contains two statuses, namely <135/85 (representing systolic/diastolic blood pressure) and no measurement. This is intended to remind the user having high blood pressure must regularly measure their own blood pressure. The medical history record deduction unit 6 presets a plurality of range values of disease 614, each range value of disease 614 presets the deduction value 42 of the corresponding disease. After the data input to the breakdown field 612 is compared with the comparison standard value 41, the medical history deduction field 613 generates a matched deduction value 42, and the calculation module 2 generates a numerical value according to the generated deduction value 42 added to the total health credit limit 3. However, based on the selection of the above two statuses, when the user selects <135/85 (representing systolic blood pressure/diastolic blood pressure), the medical history deduction column 33 will measure according to the settings, and increase the value of the total health credit limit 3. When the measured result is not listed in the standard comparison value 25, the value of the total health credit limit 3 will also be added. On the contrary, when no measurement is selected, the value of the total health credit limit 3 will be directly added. Therefore, people with chronic diseases can use the present invention to check himself regularly by inputting the chronic diseases that the user has, otherwise the total health credit limit 3 will add up and the value of the total health credit limit 3 gradually decrease.

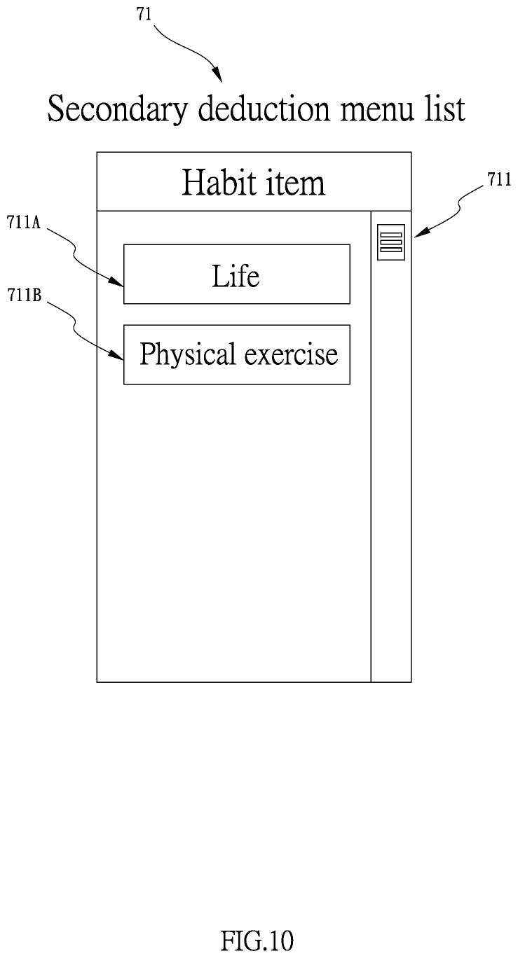

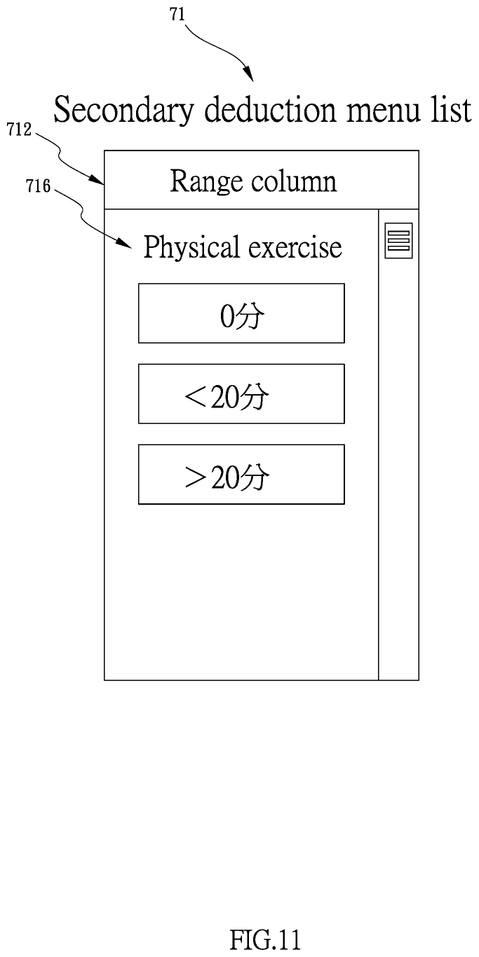

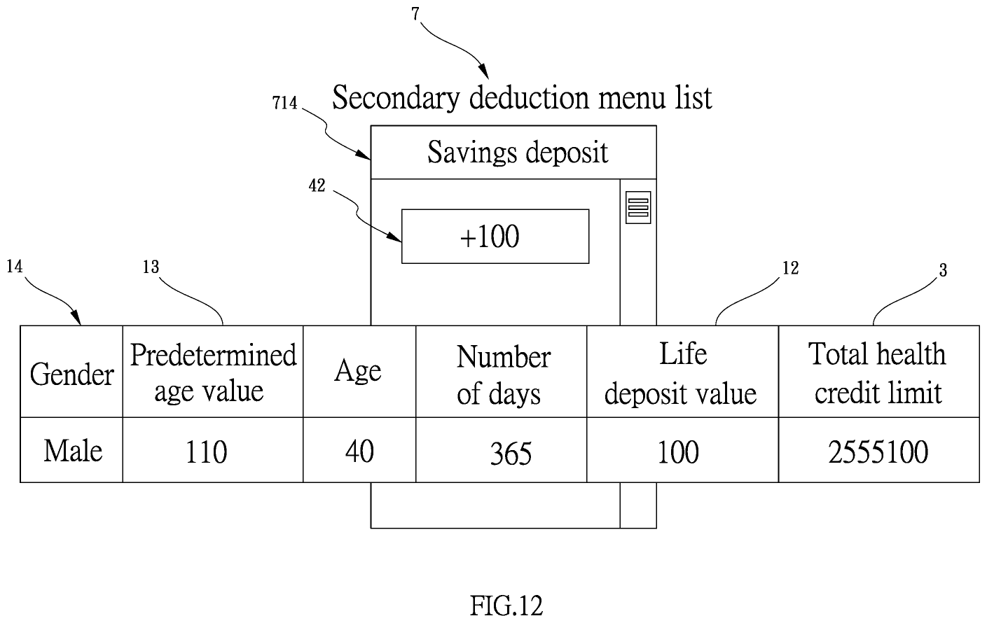

The user having chronic diseases also needs to pay attention of everyday living habit. The deduction module 4 comprises a secondary record deduction unit 7 which comprises a secondary deduction menu list 71. The secondary deduction menu list 71 has a habit item field 711, a range field 712, a savings deduction field 713 and a savings deposit field 714. The habit item field 711 is provided for inputting the user's living habit. When entering into the habit item field 711, the habit item field 711 comprises a life column 711A and a physical exercise column 711B. The life column 711A in the habit item field 711 includes a plurality of life menus 715. Each life menu 715 comprises options of eating, smoking, and drinking. The physical exercise column 711B lists a plurality of physical exercise menus 716, and each physical exercise menu 716 comprises the options of 0 minute, <20 minutes, and >20 minutes. When the user chooses the option of >20 minutes in the physical exercise menu 716, the range field 712 generates a quality range column 717 for according to the living habit of the habit item field 711. The standard numerical value of the comparison standard value 41 is a constant built in the health savings management system 1. After the quality range column 717 is selected from the range filed 712, the quality range column 717 is compared with the comparison standard value 41. After comparison, the savings deposit field 714 generates 100 dollars. The increase of the 100 dollars of the total health credit limit 3 is displayed in the output part 102. On the contrary, when the user chooses the option of 0 minute in the physical exercise menu 716, the savings deduction field 713 generates a minus 100 dollars, and 100 dollars are deducted from the total health credit limit 3. By this way, the user is reminded to pay more attention to smoking, drinking, eating and exercise as shown in FIGS. 9 to 12.

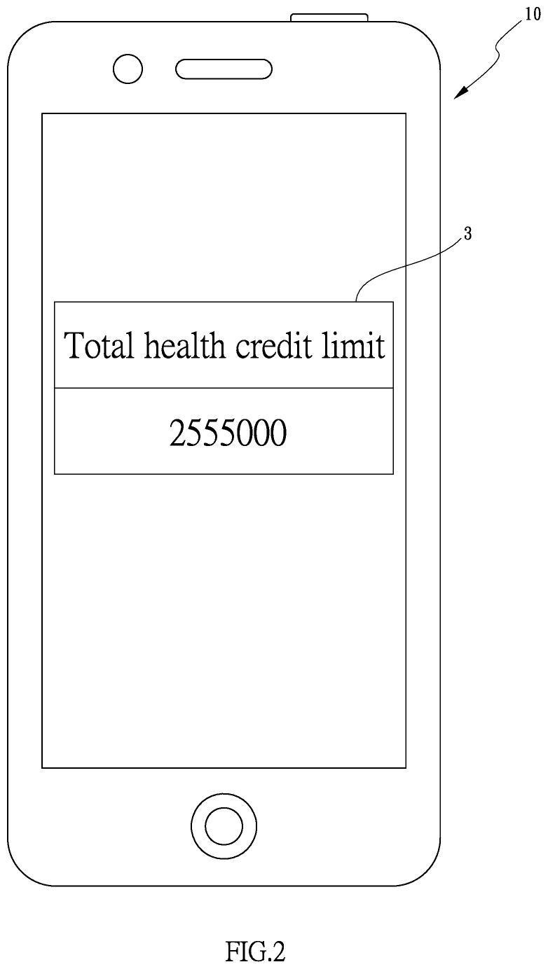

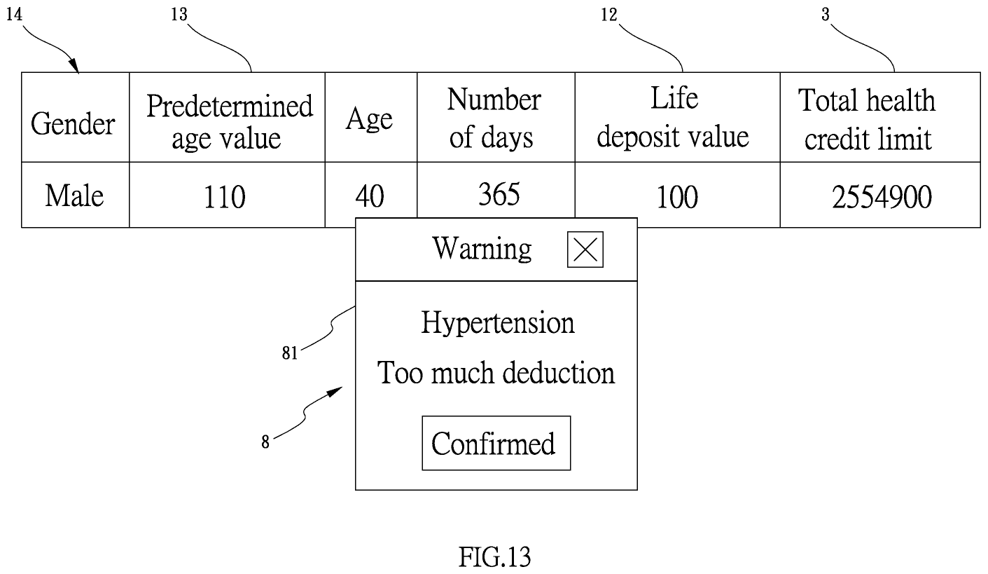

The health savings management system 1 comprises a warning module 8. When the total health credit limit 3 and the predetermined age 13 are deducted, a warning window 81 pops up to remind the user to check his/her so as to take action to consult doctors and to avoid chronic disease. Please refer to FIG. 13, the health savings management system 1 is built in the mobile device 10. The mobile device 10 can be a smart phone or a tablet. As shown in FIG. 2, by using the health savings management system 1 through the mobile device 10, the identity record module 11, the health deduction unit 5, the medical history record deduction unit 6, the secondary record deduction units 7 and the warning module 8 built in the health savings management system 1 are operated to allow the user to know his/her health condition via the sum of the digitalized money of the total health credit limit 3.

While we have shown and described the embodiment in accordance with the present invention, it should be clear to those skilled in the art that further embodiments may be made without departing from the scope of the present invention.

What is claimed is:

1. A health savings management system (1) installed and downloaded to a mobile device (10), the mobile device (10) having an input part (101) and an output part (102), the health savings management system (1) comprising:
an identity recording module (11) for recording gender, age and life deposit value (12) input through the input part (101), the identity recording module (11) having a built-in predetermined age (13);
a calculation module (2) for performing a calculation based on the predetermined age (13) and the gender, age and life deposit value (12) input to the mobile device (10) to obtain a total health credit limit (3) and to display the total health credit limit (3) on the output part (102), and
a deduction module (4) having a built-in and predetermined comparison standard value (41), the deduction module (4) recording physical condition, sickness, dietary habit and rest input by the user through the input part (101), the input physical condition, sickness, dietary habit and rest being compared with the comparison standard value (41) so as to generate a deduction value (42), the calculation module (2) adding up the predetermined age (13) and the total health credit limit (3) to change the numerical value of the total health credit limit (3) in response to the deduction value (42) generated by the deduction module (4);
wherein, the user's physical health is expressed in terms of money by the health savings management system (1) so that the user understands her/his physical condition and pays attention to and strengthen healthcare.

2. The health savings management system as claimed in claim 1, wherein the identity recording module (11) comprises an input record list (14), the input record list (14) comprises a plurality of input fields (141), each input field (141) is filled with gender, and age, a number of days and a life deposit value (12) to be calculated, one of the input fields (141) implies age which refers to the user's age, another input field (141) is the number of days which refers to the number of days required to be managed and recorded, yet another input field (141) is the life deposit value (12) which refers to the monetary value required to be saved daily after health is converted into money, the total health credit limit (3) is calculated by (Predetermined age (13)−Age)*Number of days*Life deposit value), the life deposit value (12) and the total health credit limit (3) present health condition by virtualization monetary value, the virtualization money of the life deposit value (12) represents the user's daily health amount, the monetary value of the total health credit limit (3) represents the user's health value from the numerical value of the predetermined age (13) to the user's age.

3. The health savings management system as claimed in claim 2, wherein the deduction module (4) comprises a health deduction unit (5), the health deduction unit (5) has a deduction option list (51), the deduction option list (51) comprises a basic field (511), an item field (512), a data input field (513) and a deduction field (514), the basic field (511) is provided for inputting the user's basic physical information, the item field (512) is provided for inputting and selecting scientific names of a medical test, the data input field (513) is provided for inputting the data of the names of the medial test input in the item field (512), the standard numerical value of the comparison standard value (41) is a constant built in the health savings management system (1), the data input into the data input field (513) is compared with the comparison standard value (41), the calculation module (2) is provided for calculating the deduction value (42) generated in the deduction field (514), the deduction value (42) generated in the deduction field (514) is added to the predetermined age (13) to obtain the user's total health credit limit (3), so as to check the user's physical condition.

4. The health savings management system as claimed in claim 3, wherein the basic field (511) comprises a basic information column (511A) for inputting the user's name, age, gender, height, weight and three body measurements from the input part (101).

5. The health savings management system as claimed in claim 3, wherein the item field (512) comprises an item information column (512A), the item information column (512A) is provided for inputting a scientific name of a specific sickness of the user's medical health examination by the input part (101), the item information column (512A) has a plurality of options including BMI, cholesterol, triglycerides, low density lipoprotein cholesterol, high density lipoprotein cholesterol and glomerular filtration rate.

6. The health savings management system as claimed in claim 3, wherein the data input field (513) comprises a data range column (513A), the user selects a range that matches with the data range column (513A) bases on the selected scientific name of a specific sickness.

7. The health savings management system as claimed in claim 3, wherein the health deduction unit (5) presets a plurality of range values (52), each range value (52) presets the corresponding deduction value (42), after the data range column (513A) selected from the data input field (513) is compared with the comparison standard value (41), the calculation module (2) determines if the numerical value determining the aforementioned comparison which falls within any range of each range value (52), the calculation module (2) adds the predetermined age (13) to the deduction value (42) to form the maximum value of the user's predetermined age (13).

8. The health savings management system as claimed in claim 1, wherein the deduction module (4) comprises a medical history record deduction unit (6), the medical history record deduction unit (6) comprises a sickness deduction menu list (61), the sickness deduction menu list (61) comprises a disease name field (611), a breakdown field (612) and a medical history deduction field (613), the disease name field (611) is provided for inputting the user's chronic disease, the breakdown field (612) is provided for inputting the user's corresponding examination data according to the chronic disease selected from the disease name field (611), the standard numerical value of the comparison standard value (41) is a constant built in the health savings management system (1), the examination data input to the breakdown field (612) is compared with the comparison standard value (41), the calculation module (2) performs the computation to further generate a deduction value (42) in the medical history deduction field (613), the deduction value (42) generated in the medical history deduction field (613) is added to the total health credit limit (3) to form the maximum value.

9. The health savings management system as claimed in claim 8, wherein the disease name field (611) comprises a medical history scientific name column (611A), the medical history scientific name column (611A) is provided for inputting the user's disease names by the input part (101), the medical history scientific name column (611A) comprises a disease name of hypertension, diabetes, coronary heart disease or stroke.

10. The health savings management system as claimed in claim 8, wherein the breakdown field (612) is provided for selecting a numerical value range column (612A) according to the sickness selected from the disease name field (611), the user selects the matched numerical value range column (612A) according to the data of the health examination.

11. The health savings management system as claimed in claim 8, wherein the medical history record deduction unit (6) presets a plurality of range value of disease (614), each range value of disease (614) presets the deduction value (42) of the corresponding disease, after the data input to the breakdown field (612) is compared with the comparison standard value (41), the medical history deduction field (613) generates a matched deduction value (42), and the calculation module (2) generates a numerical value according to the generated deduction value (42) added to the total health credit limit (3).

12. The health savings management system as claimed in claim 1, wherein the deduction module (4) comprises a secondary record deduction unit (7), the secondary record deduction unit (7) comprises a secondary deduction menu list (71), the secondary deduction menu list (71) has a habit item field (711), a range field (712), a savings deduction field (713) and a savings deposit field (714), the habit item field (711) is provided for inputting the user's living habit, the range field (712) generates a quality range column (717) in response to the living habit in the habit item field (711), the standard numerical value of the comparison standard value (41) is a constant built in the health savings management system (1), after the quality range column (717) selected from and matched with the range filed (712) is compared with the comparison standard value (41) to generate a deduction value (42) in the savings deduction field (713) and the savings deposit field (714), the deduction value (42) generated in the savings deduction field (713) and the savings deposit field (714) are added to the total health credit limit (3) to form a maximum value.

13. The health savings management system as claimed in claim 12, wherein the habit item field (711) comprises a life column (711A) and a physical exercise column (711B), the user selects the life column (711A) and the physical exercise column (711B) according to the living habit to adjust the numerical value of the total health credit limit (3) in order to check the user's physical health care condition by the user.

14. The health savings management system as claimed in claim 13, wherein the life column (711A) in the habit item field (711) lists a plurality of life menus (715), each life menu (715) comprises options of eating, smoking, and drinking; the physical exercise column (711B) lists a plurality of physical exercise menus (716), and each physical exercise menu (716) comprises the options of 0 minute, <20 minutes, and >20 minutes, and the user makes a choice according to the user's own condition and knows about the user's physical condition by the change of the total health credit limit (3).

15. The health savings management system as claimed in claim 1, wherein the health savings management system (1) comprises a warning module (8), when the total health credit limit (3) and the numerical value of the predetermined age (13) are deducted, a warning window (81) pops up, the user is reminded to check physical condition by the pop-up warning window (81).
